(19) United States
(12) Patent Application Publication
(10) Pub. No.: US 2020/0375574 A1
(43) Pub. Date: Dec. 3, 2020

(54) ULTRASOUND IMAGING SPATIAL COMPOUNDING METHOD AND SYSTEM

(71) Applicant: VINNO TECHNOLOGY (SUZHOU) CO., LTD., Suzhou City (CN)

(72) Inventors: TAO LING, Suzhou City (CN); RUI MA, Suzhou City (CN)

(21) Appl. No.: 16/882,713

(22) Filed: May 25, 2020

(30) Foreign Application Priority Data
May 28, 2019 (CN) 201910448919.X

(51) Int. Cl. A61B 8/08

(52) U.S. Cl. CPC A61B 8/5253

(57) ABSTRACT

The present invention provides an ultrasound imaging spatial compounding method and system. The method includes: setting receive lines at different deflection angles in a position of a transmitted beam where each scanning is performed; obtaining the receive lines at the different angles through beamforming; after all positions are scanned, enabling the receive lines at the same deflection angle to form a frame of image at the angle, using one of a plurality of frames of image at the different deflection angles as a basic image, and transforming the remaining frames of image except the basic image into images having the same coordinate system as the basic image; and performing spatial compounding on the plurality of frames of image in the same coordinate system to obtain a compound image for output. The present invention does not affect the temporal resolution of imaging, thereby avoiding image lagging and trailing phenomena.

### TECHNICAL FIELD

The present invention relates to the field of medical ultrasound diagnostic imaging, and in particular to an ultrasound imaging spatial compounding method and system.

### BACKGROUND

Ultrasound imaging has various advantages such as noninvasiveness, real-time performance, convenient operations, and low prices, and therefore becomes one of the most widely clinically applied diagnostic tools. During ultrasound imaging, a probe transmits a focused ultrasound beam. Elements of the probe receive an ultrasound echo signal, and amplification and filtering are performed in each channel. Beamforming is performed on a channel-level signal to obtain a radio frequency (RF) signal. The foregoing scanning process is repeated until a frame of RF signal with a particular linear density is obtained. The RF signal is demodulated and filtered to obtain a quadrature (IQ) signal. The IQ signal is processed to obtain an image. The image is post-processed to be eventually displayed on a display for output.

The most frequently used functional mode of ultrasound imaging is a two-dimensional (2D) black-and-white (B) mode. The B mode depends on the amplitude of an ultrasound echo signal for imaging. The 2D structure and form information of tissue are acquired. When the echo signal is more intense, a corresponding image pixel has a larger gray level value, or otherwise, the gray level value is smaller. Limited by physical properties of ultrasound waves and an imaging method, “speckle” noise inevitably occurs in imaging in the B mode, and there are requirements in signal-to-noise ratio (SNR) and contrast.

A spatial compounding technology is a common processing method in imaging in the B mode, which utilizes electronic delay to deflect a scanning sound beam so as to obtain images at different angles. Pixel values at a same geometric spatial position on a plurality of frames of image at different angles are then weighted and superposed to obtain a spatially compounded image. The spatial compounding technology can effectively reduce “speckle” noise, so that an image of uniform tissue is smoother and finer, and SNR and contrast of the image can further be significantly improved to facilitate diagnosis by a clinical physician. In addition, information at different angles can be obtained through scans at different deflection angles to detect interfaces in different directions, and more detailed image information and better interface continuity are achieved after spatial compounding. Another important application of the spatial compounding technology is display enhancement with a puncture needle. By means of deflection scanning with spatial compounding, an incident sound beam is made as perpendicular as possible to the surface of a puncture needle, so as to obtain an intense surface image of the puncture needle.

In an existing technical solution, an electronic delay is utilized to control transmit and receive beams across the surface of a probe to deflect at a certain angle until scanning is completed and a frame of complete image at the angle is obtained. The foregoing process is repeated to obtain an image at other angle(s). An existing spatial compounding technology is usually performed in a manner of “rolling processing”. For example, N frames of image are spatially compounded. One frame of image at a different angle is obtained during each scanning. The latest frame of image obtained each time and the previous N−1 frames of image are spatially compounded. The process is repeated to implement real-time spatial compounding imaging.

FIG. 1 shows a commonly used method in the prior art. Sequential scanning is performed to obtain each frame of image at different deflection angles. The images at all angles are then superposed according to a particular weighting coefficient to obtain a compound image. An area 1 is an overlap area of three frames of image, areas 2 are overlap areas of two frames of image, and areas 3 do not overlap and are eventually not displayed for output.

However, the prior art can satisfy a requirement of real-time performance, and frame frequency is not reduced. But, when there is a large angle or many angles in compounding, severe lagging and trailing phenomena occur in an image, that is, the temporal resolution of the image is reduced.

### SUMMARY

To resolve the foregoing technical problem, an objective of the present invention is to provide an ultrasound imaging spatial compounding method and system.

To achieve one of the foregoing inventive objectives, an implementation of the present invention provides an ultrasound imaging spatial compounding method, where the method includes: setting receive lines at different deflection angles in a position of a transmitted beam where each scanning is performed, where the receive lines are obtained through deflection at a plurality of angles based on receive lines in a normal direction of a probe and with a depth position of a transmission focus as a reference point;

obtaining the receive lines at the different angles through beamforming, where a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe includes a type and a geometrical parameter of the probe;

after all positions are scanned, enabling the receive lines at the same deflection angle to form a frame of image at the angle,

using one of a plurality of frames of image at the different deflection angles as a basic image, and transforming the remaining frames of image except the basic image into images having the same coordinate system as the basic image; and

performing spatial compounding on the plurality of frames of image in the same coordinate system to obtain a compound image for output.

As a further improvement to an implementation of the present invention, if the type of the probe is a linear array probe, the wavefront delay of the transmitted beam is represented as:

wavefront_delay(a)=(focus−focus/cos(a))/c,

where focus represents the depth of the transmission focus, c represents a sound velocity, and a represents a deflection angle of the receive lines relative to the receive lines in the normal direction of the probe.

As a further improvement to an implementation of the present invention, if the type of the probe is a curved array probe, the wavefront delay of the transmitted beam is represented as:

wavefront_delay=(focus−ROC/sin(a)*sin(a sin((ROC+focus)/ROC*sin(a))−a))/c,

where focus represents the depth of the transmission focus, ROC represents the radius of curvature of the probe, c represents a sound velocity, and a represents a deflection angle of the receive lines relative to the receive lines in the normal direction of the probe.

As a further improvement to an implementation of the present invention, the “using one of a plurality of frames of image at the different deflection angles as a basic image, and transforming the remaining frames of image except the basic image into images having the same coordinate system as the basic image” specifically includes:

using a frame of image of the receive lines in the normal direction of the probe as the basic image; and

by using the basic image as a reference, transforming deflected receive lines in another frame of image to the positions of the receive lines in the basic image in an interpolation and/or resampling manner, and transforming the remaining frames of image except the basic image into images having the same coordinate system as the basic image.

As a further improvement to an implementation of the present invention, the “performing spatial compounding on the plurality of frames of image in the same coordinate system to obtain a compound image for output” specifically includes:

performing spatial compounding on the plurality of frames of image corresponding to a geometric spatial position in a manner of performing one of averaging, weighted averaging, maximum finding, and median finding on gray levels of different frames to form the compound image.

To achieve one of the foregoing inventive objectives, an implementation of the present invention provides an ultrasound imaging spatial compounding system, where the system includes: a receiving setting module, configured to set receive lines at different deflection angles in a position of a transmitted beam where each scanning is performed, where the receive lines are obtained through deflection at a plurality of angles based on receive lines in a normal direction of a probe and with a depth position of a transmission focus as a reference point;

a beamforming module, configured to obtain the receive lines at the different angles through beamforming, where a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe includes a type and a geometrical parameter of the probe;

a coordinate transformation module, configured to: after all positions are scanned, enable the receive lines at the same deflection angle to form a frame of image at the angle, use one of a plurality of frames of image at the different deflection angles as a basic image, and transform the remaining frames of image except the basic image into images having the same coordinate system as the basic image; and

an image compounding output module, configured to perform spatial compounding on the plurality of frames of image in the same coordinate system to obtain a compound image for output.

As a further improvement to an implementation of the present invention, if the type of the probe is a linear array probe, the wavefront delay of the transmitted beam obtained by the beamforming module is represented as:

wavefront_delay(a)=(focus−focus/cos(a))/c,

where focus represents the depth of the transmission focus, c represents a sound velocity, and a represents a deflection angle of the receive lines relative to the receive lines in the normal direction of the probe.

As a further improvement to an implementation of the present invention, if the type of the probe is a curved array probe, the wavefront delay of the transmitted beam obtained by the beamforming module is represented as:

wavefront_delay=(focus−ROC/sin(a)*sin(a sin((ROC+focus)/ROC*sin(a))−a))/c,

where focus represents the depth of the transmission focus, ROC represents the radius of curvature of the probe, c represents a sound velocity, and a represents a deflection angle of the receive lines relative to the receive lines in the normal direction of the probe.

As a further improvement to an implementation of the present invention, the coordinate transformation module is specifically configured to:

use a frame of image of the receive lines in the normal direction of the probe as the basic image; and

by using the basic image as a reference, transform deflected receive lines in another frame of image to the positions of the receive lines in the basic image in an interpolation and/or resampling manner, and transform the remaining frames of image except the basic image into images having the same coordinate system as the basic image.

As a further improvement to an implementation of the present invention, the image compounding output module is specifically configured to:

perform spatial compounding on the plurality of frames of image corresponding to a geometric spatial position in a manner of performing one of averaging, weighted averaging, maximum finding, and median finding on gray levels of different frames to form the compound image.

Compared with the prior art, the beneficial effects of the present invention are as follows: the ultrasound imaging spatial compounding method and system according to the present invention do not affect the temporal resolution of imaging, thereby avoiding image lagging and trailing phenomena in the prior art.

### BRIEF DESCRIPTION OF THE DRAWINGS

FIG. 1 is a schematic structural diagram of an image compounding method mentioned in the background of the present invention;

FIG. 2 is a schematic flowchart of an ultrasound imaging spatial compounding method according to an implementation of the present invention;

FIG. 3 is a schematic diagram of comparison between deflection of a transmitted beam and deflection of a receive beam in a specific example of the present invention;

FIG. 4 is a schematic diagram of a display effect of a wavefront of a transmitted beam in a specific example of the present invention;

FIG. 5 is a schematic diagram of a wavefront delay of a transmitted beam of a linear array probe in a specific example of the present invention;

FIG. 6 is a schematic diagram of a wavefront delay of a transmitted beam of a curved array probe in a specific example of the present invention;

FIG. 7 is a schematic diagram of an effect of transforming image coordinates in a specific example of the present invention; and

FIG. 8 is a schematic modular diagram of an ultrasound imaging spatial compounding system according to an implementation of the present invention.

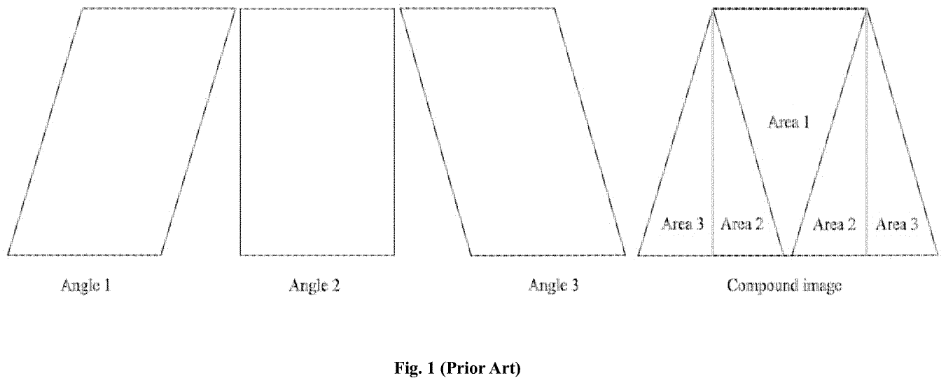

### DETAILED DESCRIPTION

The present invention is described below in detail with reference to specific implementations shown in the accompanying drawings. However, these implementations do not limit the present invention. Variations made to the structure, method or function by a person of ordinary skill in the art according to these implementations all fall within the protection scope of the present invention.

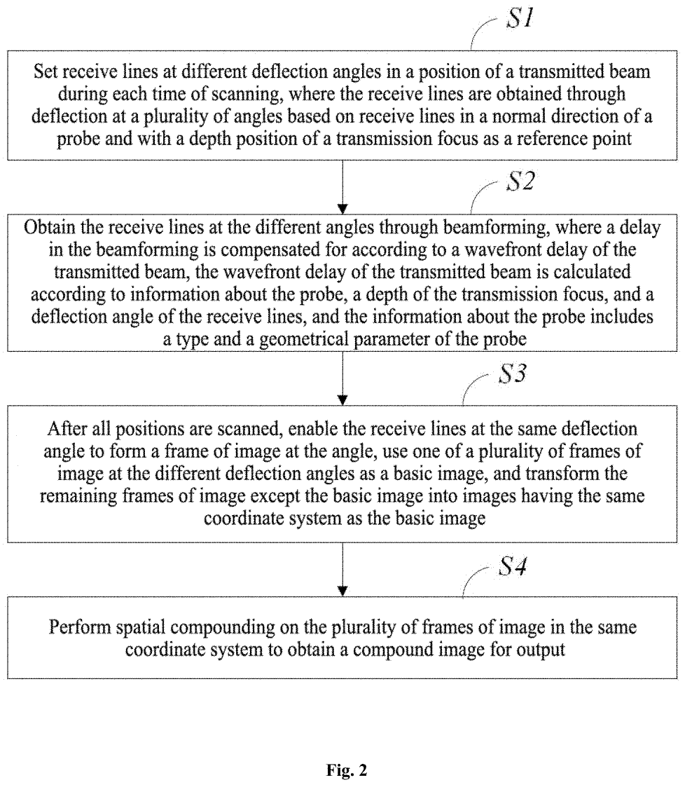

As shown in FIG. 2, an implementation of the present invention provides an ultrasound imaging spatial compounding method. The method includes the following steps.

S1: Set receive lines at different deflection angles in a position of a transmitted beam where each scanning is performed, where the receive lines are obtained through deflection at a plurality of angles based on receive lines in a normal direction of a probe and with a depth position of a transmission focus as a reference point.

S2: Obtain the receive lines at the different angles through beamforming, where a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe includes a type and a geometrical parameter of the probe.

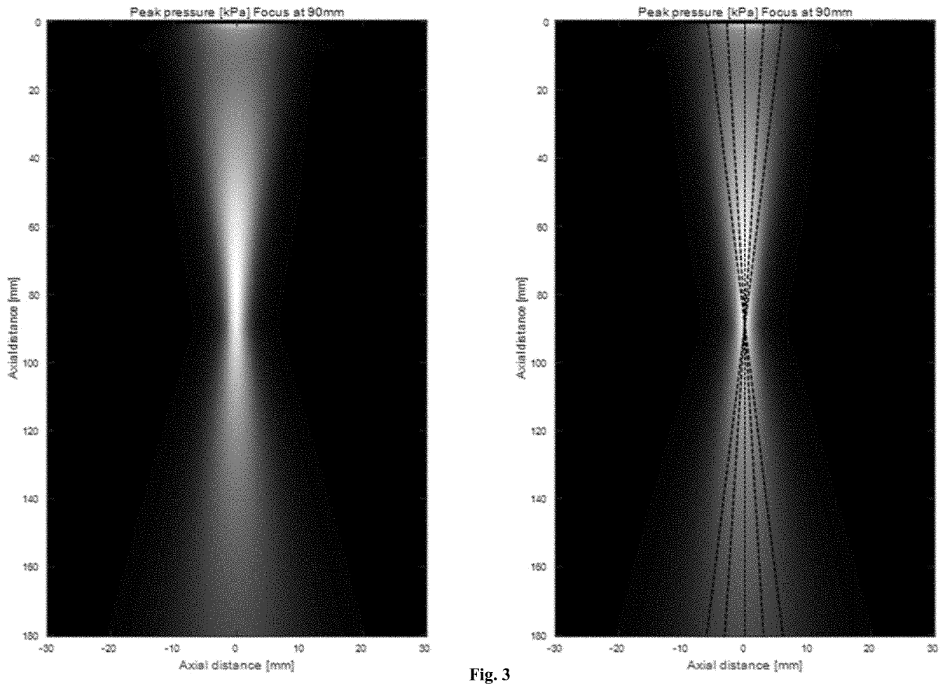

Referring to FIG. 3, when a plurality of elements of an ultrasound probe implement focused transmission in an electronic delay manner, as shown in the left figure of FIG. 3, a sound field of the transmitted beam usually has an “hourglass” shape. The sound field gradually converges in front of a focus, and the sound field gradually diverges behind the focus. Therefore, the sound field is narrowest at the focus. In the present invention, a receive beam is deflected with a depth position of a transmission focus as a reference point, to enable the arrangement of the receive lines to have a consistent form with a transmission sound field, so that the coverage of the transmission sound field can be fully used to acquire more useful signals. Referring to the right figure of FIG. 3, to enable the transmission sound field to cover a larger area to obtain receive lines at a larger deflection angle, in a preferred implementation of the present invention, a transmit aperture is appropriately increased or a transmit apodization is appropriately reduced. That is, when an effect of spatial compounding is weakened because the transmitted beam is not deflected, the deflection angle of the receive lines may be appropriately increased to compensate for the defect. Details are not further described herein.

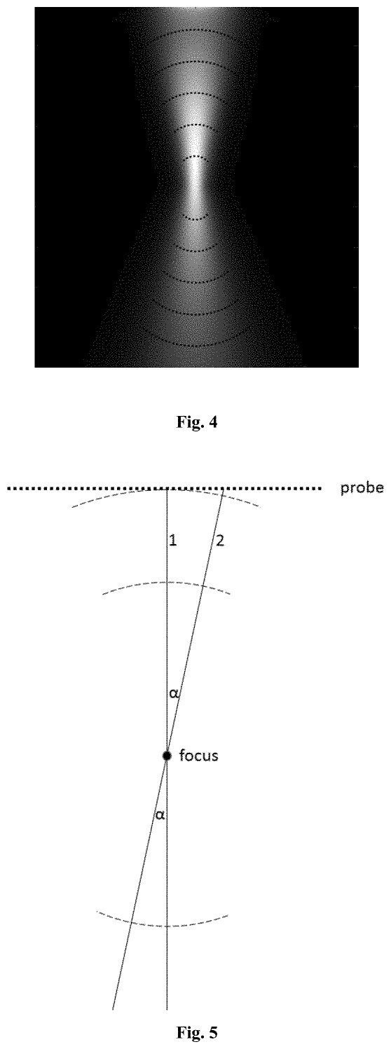

Further, referring to FIG. 4, during the implementation of the present invention, because the angle of the transmitted beam is not deflected, and only the receive lines are deflected at a plurality of angles, receive beamforming is different from a conventional manner mainly in that a time difference between a wavefront of the transmitted beam on receive lines at different angles needs to be considered for a delay of beamforming. The wavefront of the transmitted beam gradually converges from the surface of the probe toward the position of the focus, and then gradually diverges outward from the position of the focus. In an ideal case, the wavefront of the transmit signal is a concentric circle with the position of the focus of transmission being the center of circle.

Referring to FIG. 5, in a preferred implementation of the present invention, the type of the ultrasound probe is a linear array probe, and the wavefront delay of the transmitted beam is represented as:

wavefront_delay(a)=(focus−focus/cos(a))/c,

where focus represents the depth of the transmission focus, c represents a sound velocity, and a represents a deflection angle of the receive lines relative to the receive lines in the normal direction of the probe.

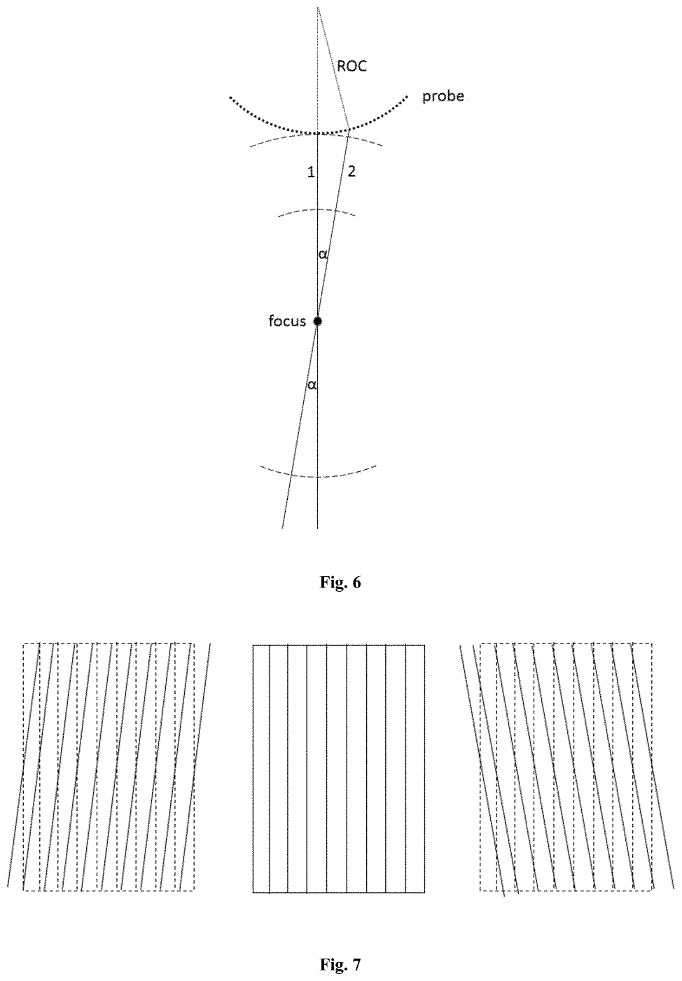

Referring to FIG. 6, in a preferred implementation of the present invention, the type of the ultrasound probe is a curved array probe, and the wavefront delay of the transmitted beam is represented as:

wavefront_delay=(focus−ROC/sin(a)*sin(a sin((ROC+focus)/ROC*sin(a))−a))/c,

where focus represents the depth of the transmission focus, ROC represents the radius of curvature of the probe, c represents a sound velocity, and a represents a deflection angle of the receive lines relative to the receive lines in the normal direction of the probe.

It should be noted that for a phased array probe, because the size of the probe is relatively small, an application space of the probe is relatively small. Therefore, specific application of the probe is no longer described in detail. However, it may be understood that a solution of using a linearly controlled array probe for a spatial compounding technology under the concept of the present invention still falls within the protection scope of the present invention. Details are not further described herein.

A beamforming computation method is a mature technical solution known to a person skilled in the art. Therefore, a beamforming technology is not further described.

Further, the method further includes the following step.

S3: After all positions are scanned, enable the receive lines at the same deflection angle to form a frame of image at the angle, use one of a plurality of frames of image at the different deflection angles as a basic image, and transform the remaining frames of image except the basic image into images having the same coordinate system as the basic image.

In a preferred implementation of the present invention, step S3 specifically includes the following steps:

M1: Use a frame of image of the receive lines in the normal direction of the probe as the basic image.

M2: By using the basic image as a reference, transform deflected receive lines in another frame of image to the positions of the receive lines in the basic image in an interpolation and/or resampling manner, and transform the remaining frames of image except the basic image into images having the same coordinate system as the basic image.

During specific application of the present invention, only one of a plurality of frames of image obtained after beamforming processing, that is, a frame of image of the receive lines in the normal direction of the probe, is a conventional image, and the remaining frames of image are all deflection images having a deflection angle relative to the basic image, that is, images obtained after the receive lines are deflected at the position of the transmission focus by a certain angle from the normal direction.

Referring to FIG. 7, for the three frames of image in the figure, an image a without deflection is a basic image, and both an image b with deflection to the right and an image c with deflection to the left require coordinate transformation relative to the image a, so that the image b and image c are transformed to have the same position as the image a.

In a specific example, receive lines in a conventional frame of image a are used as a reference, and deflected receive lines (solid lines shown in the figure) in images b and c are transformed through interpolation and/or resampling to positions (dotted lines shown in the figure) corresponding to the receive lines in the image a. Therefore, the images b and c are transformed to have the same coordinate system as the image a, so that a same pixel in the transformed image represents information of the same position.

Further, the method further includes the following step. S4: Perform spatial compounding on the plurality of frames of image in the same coordinate system to obtain a compound image for output.

In a preferred implementation of the present invention, spatial compounding is performed on a plurality of frames of image corresponding to a geometric spatial position in a manner of performing averaging, weighted averaging, maximum finding, median finding or the like on gray levels of different frames to form the compound image.

In a specific implementation of the present invention, in consideration of that images at different deflection angles have different amounts of information, a method of performing weighted averaging according to a particular weight coefficient is used to perform spatial compounding on the plurality of frames of image at different angles. Details are not further described herein.

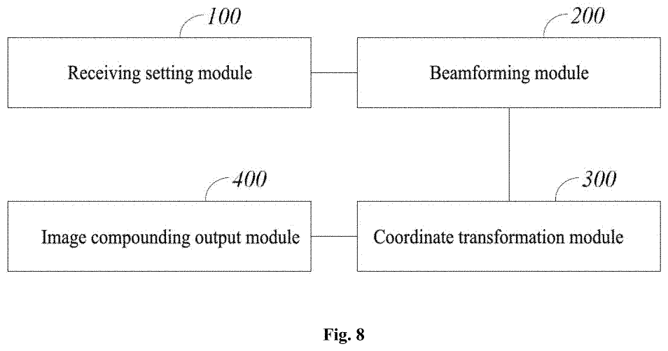

Referring to FIG. 8, an implementation of the present invention provides an ultrasound imaging spatial compounding system. The system includes a receiving setting module 100, a beamforming module 200, a coordinate transformation module 300, and an image compounding output module 400.

The receiving setting module 100 is configured to set receive lines at different deflection angles in a position of a transmitted beam where each scanning is performed, where the receive lines are obtained through deflection at a plurality of angles based on receive lines in a normal direction of a probe and with a depth position of a transmission focus as a reference point.

The beamforming module 200 is configured to obtain the receive lines at the different angles through beamforming, where a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe includes a type and a geometrical parameter of the probe.

Referring to FIG. 3, when a plurality of elements of an ultrasound probe implement focused transmission in an electronic delay manner, as shown in the left figure of FIG. 3, a sound field of the transmitted beam usually has an “hourglass” shape. The sound field gradually converges in front of a focus, and the sound field gradually diverges behind the focus. Therefore, the sound field is narrowest at the focus. In the present invention, a receive beam is deflected with a depth position of a transmission focus as a reference point, to enable the arrangement of the receive lines to have a consistent form with a transmission sound field, so that the coverage of the transmission sound field can be fully used to acquire more useful signals. Referring to the right figure of FIG. 3, to enable the transmission sound field to cover a larger area to obtain receive lines at a larger deflection angle, in a preferred implementation of the present invention, a transmit aperture is appropriately increased or a transmit apodization is appropriately reduced. That is, when an effect of spatial compounding is weakened because the transmitted beam is not deflected, the deflection angle of the receive lines may be appropriately increased to compensate for the defect. Details are not further described herein.

Further, referring to FIG. 4, during the implementation of the present invention, because the angle of the transmitted beam is not deflected, and only the receive lines are deflected at a plurality of angles, receive beamforming is different from a conventional manner mainly in that a time difference between a wavefront of the transmitted beam on receive lines at different angles needs to be considered for a delay of beamforming. The wavefront of the transmitted beam gradually converges from the surface of the probe toward the position of the focus, and then gradually diverges outward from the position of the focus. In an ideal case, the wavefront of the transmit signal is a concentric circle with the position of the focus of transmission being the center of circle.

Referring to FIG. 5, in a preferred implementation of the present invention, the type of the ultrasound probe is a linear array probe, and the wavefront delay of the transmitted beam obtained by the beamforming module 200 is represented as:

wavefront_delay(a)=(focus−focus/cos(a))/c,

where focus represents the depth of the transmission focus, c represents a sound velocity, and a represents a deflection angle of the receive lines relative to the receive lines in the normal direction of the probe.

Referring to FIG. 6, in a preferred implementation of the present invention, the type of the ultrasound probe is a curved array probe, and the wavefront delay of the transmitted beam obtained by the beamforming module 200 is represented as:

wavefront_delay=(focus−ROC/sin(a)*sin(a sin((ROC+focus)/ROC*sin(a))−a))/c,

where focus represents the depth of the transmission focus, ROC represents the radius of curvature of the probe, c represents a sound velocity, and a represents a deflection angle of the receive lines relative to the receive lines in the normal direction of the probe.

It should be noted that for a phased array probe, because the size of the probe is relatively small, an application space of the probe is relatively small. Therefore, specific application of the probe is no longer described in detail. However, it may be understood that a solution of using a linearly controlled array probe for a spatial compounding technology under the concept of the present invention still falls within the protection scope of the present invention. Details are not further described herein.

The coordinate transformation module 300 is configured to: after all positions are scanned, enable the receive lines at the same deflection angle to form a frame of image at the angle, use one of a plurality of frames of image at the different deflection angles as a basic image, and transform the remaining frames of image except the basic image into images having the same coordinate system as the basic image.

The coordinate transformation module 300 in a preferred implementation of the present invention is specifically configured to: use a frame of image of the receive lines in the normal direction of the probe as the basic image; and by using the basic image as a reference, transform deflected receive lines in another frame of image to the positions of the receive lines in the basic image in an interpolation and/or resampling manner, and transform the remaining frames of image except the basic image into images having the same coordinate system as the basic image.

During specific application of the present invention, only one of a plurality of frames of image obtained after beamforming processing, that is, a frame of image of the receive lines in the normal direction of the probe, is a conventional image, and the remaining frames of image are all deflection images having a deflection angle relative to the basic image, that is, images obtained after the receive lines are deflected at the position of the transmission focus by a certain angle from the normal direction.

Referring to FIG. 7, for the three frames of image in the figure, an image a without deflection is a basic image, and both an image b with deflection to the right and an image c with deflection to the left require coordinate transformation relative to the image a, so that the image b and image c are transformed to have the same position as the image a.

In a specific example, receive lines in a conventional frame of image a are used as a reference, and deflected receive lines (solid lines shown in the figure) in the images b and c are transformed through interpolation and/or resampling to positions (dotted lines shown in the figure) corresponding to the receive lines in the image a. Therefore, the images b and c are transformed to have the same coordinate system as the image a, so that a same pixel in the transformed image represents information of the same position.

The image compounding output module 400 is configured to perform spatial compounding on the plurality of frames of image in the same coordinate system to obtain a compound image for output.

In a preferred implementation of the present invention, the image compounding output module 400 is configured to perform spatial compounding on the plurality of frames of image corresponding to a geometric spatial position in a manner of performing averaging, weighted averaging, maximum finding, median finding or the like on gray levels of different frames to form the compound image.

In a specific implementation of the present invention, in consideration of that images at different deflection angles have different amounts of information, a method of performing weighted averaging according to a particular weight coefficient is used to perform spatial compounding on the plurality of frames of image at different angles. Details are not further described herein.

In summary, the ultrasound imaging spatial compounding method and system of the present invention do not require sound beam deflection in a transmission stage.

Instead, by using physical properties of a transmitted beam, receive lines at different deflection angles are set in a position of the transmitted beam where each scanning is performed, so that a plurality of receive lines at a different angle are obtained during a single time of transmission and a plurality of frames of image at the different angle are obtained within the imaging time of a single frame, and weighted superposition is then performed on the plurality of frames of image at different angles according to a particular weight coefficient to obtain a spatially compounded image. The technology in the present invention does not affect the temporal resolution of imaging, thereby avoiding image lagging and trailing phenomena in the prior art.

For ease of description, in the description of the foregoing apparatus, various functional modules of the apparatus are described. Certainly, during the implementation of the present invention, the functions of various modules may be implemented in the same one or more pieces of software and/or hardware.

The described apparatus implementation is merely exemplary. The modules described as separate parts may or may not be physically separated, and parts shown as modules may or may not be physical modules, which may be located in one position, or may be distributed on a plurality of network modules. Some or all of the modules may be selected according to actual needs to achieve the objectives of the solutions of the implementations. Persons of ordinary skill in the art may understand implement the implementations without creative efforts.

It should be understood that although the specification is described according to the implementations, each implementation does not necessarily include only one independent technical solution. The description manner of the specification is only used for clarity, and a person skilled in the art should consider the specification as a whole. The technical solutions in the implementations may be appropriately combined to constitute other implementations comprehensible to a person skilled in the art.

A series of detailed descriptions listed above are only specific descriptions of feasible implementations of the present invention, but are not used to limit the protection scope of the present invention. Any equivalent implementation or variation made without departing from the technical spirit of the present invention shall fall within the protection scope of the present invention.

What is claimed is:

1. An ultrasound imaging spatial compounding method, wherein the method comprises:
setting receive lines at different deflection angles in a position of a transmitted beam where each scanning is performed, wherein the receive lines are obtained through deflection at a plurality of angles based on receive lines in a normal direction of a probe and with a depth position of a transmission focus as a reference point;
obtaining the receive lines at the different angles through beamforming, wherein a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe comprises a type and a geometrical parameter of the probe;
after all positions are scanned, enabling the receive lines at the same deflection angle to form a frame of image at the angle, using one of a plurality of frames of image at the different deflection angles as a basic image, and transforming the remaining frames of image except the basic image into images having the same coordinate system as the basic image; and
performing spatial compounding on the plurality of frames of image in the same coordinate system to obtain a compound image for output.

2. The ultrasound imaging spatial compounding method according to claim 1, wherein if the type of the probe is a linear array probe, the wavefront delay of the transmitted beam is represented as:
wavefront_delay(a)=(focus−focus/cos(a))/c;
wherein focus represents the depth of the transmission focus, c represents a sound velocity, and a represents a deflection angle of the receive lines relative to the receive lines in the normal direction of the probe.

3. The ultrasound imaging spatial compounding method according to claim 1, wherein if the type of the probe is a curved array probe, the wavefront delay of the transmitted beam is represented as:
wavefront_delay=(focus−ROC/sin(a)*sin(a sin((ROC+focus)/ROC*sin(a))−a))/c;
wherein focus represents the depth of the transmission focus, ROC represents the radius of curvature of the probe, c represents a sound velocity, and a represents a deflection angle of the receive lines relative to the receive lines in the normal direction of the probe.

4. The ultrasound imaging spatial compounding method according to claim 1, wherein the “using one of a plurality of frames of image at the different deflection angles as a basic image, and transforming the remaining frames of image except the basic image into images having the same coordinate system as the basic image” specifically comprises:
using a frame of image of the receive lines in the normal direction of the probe as the basic image; and
by using the basic image as a reference, transforming deflected receive lines in another frame of image to the positions of the receive lines in the basic image in an interpolation and/or resampling manner, and transforming the remaining frames of image except the basic image into images having the same coordinate system as the basic image.

5. The ultrasound imaging spatial compounding method according to claim 4, wherein:
the “performing spatial compounding on the plurality of frames of image in the same coordinate system to obtain a compound image for output” specifically comprises:
performing spatial compounding on the plurality of frames of image corresponding to a geometric spatial position in a manner of performing one of averaging, weighted averaging, maximum finding, and median finding on gray levels of different frames to form the compound image.

6. An ultrasound imaging spatial compounding system, wherein the system comprises:
a receiving setting module, configured to set receive lines at different deflection angles in a position of a transmitted beam where each scanning is performed, wherein the receive lines are obtained through deflection at a plurality of angles based on receive lines in a normal direction of a probe and with a depth position of a transmission focus as a reference point;
a beamforming module, configured to obtain the receive lines at the different angles through beamforming, wherein a delay in the beamforming is compensated for according to a wavefront delay of the transmitted beam, the wavefront delay of the transmitted beam is calculated according to information about the probe, a depth of the transmission focus, and a deflection angle of the receive lines, and the information about the probe comprises a type and a geometrical parameter of the probe;
a coordinate transformation module, configured to: after all positions are scanned, enable the receive lines at the same deflection angle to form a frame of image at the angle, use one of a plurality of frames of image at the different deflection angles as a basic image, and transform the remaining frames of image except the basic image into images having the same coordinate system as the basic image; and
an image compounding output module, configured to perform spatial compounding on the plurality of frames of image in the same coordinate system to obtain a compound image for output.

7. The ultrasound imaging spatial compounding system according to claim 6, wherein if the type of the probe is a linear array probe, the wavefront delay of the transmitted beam obtained by the beamforming module is represented as:
wavefront_delay(a)=(focus−focus/cos(a))/c;
wherein focus represents the depth of the transmission focus, c represents a sound velocity, and a represents a deflection angle of the receive lines relative to the receive lines in the normal direction of the probe.

8. The ultrasound imaging spatial compounding system according to claim 6, wherein if the type of the probe is a curved array probe, the wavefront delay of the transmitted beam obtained by the beamforming module is represented as:
wavefront_delay=(focus−ROC/sin(a)*sin(a sin((ROC+focus)/ROC*sin(a))−a))/c;
wherein focus represents the depth of the transmission focus, ROC represents the radius of curvature of the probe, c represents a sound velocity, and a represents a deflection angle of the receive lines relative to the receive lines in the normal direction of the probe.

9. The ultrasound imaging spatial compounding system according to claim 6, wherein the coordinate transformation module is specifically configured to:
use a frame of image of the receive lines in the normal direction of the probe as the basic image; and
by using the basic image as a reference, transform deflected receive lines in another frame of image to the positions of the receive lines in the basic image in an interpolation and/or resampling manner, and transform the remaining frames of image except the basic image into images having the same coordinate system as the basic image.

10. The ultrasound imaging spatial compounding system according to claim 9, wherein the image compounding output module is specifically configured to:
perform spatial compounding on the plurality of frames of image corresponding to a geometric spatial position in a manner of performing one of averaging, weighted averaging, maximum finding, and median finding on gray levels of different frames to form the compound image.
